(19) United States
(12) Patent Application Publication
(10) Pub. No.: US 2021/0261527 A1
(43) Pub. Date: Aug. 26, 2021

(54) OGA INHIBITOR COMPOUNDS

(71) Applicant: Janssen Pharmaceutica NV, Beerse (BE)

(72) Inventors: José Manuel Bartolomé-Nebreda, Toledo (ES); Andres Avellno Trabanco-Suarez, Olias del Rey (ES); Carlos Manuel Martinez-Viturro, Olias del Rey (ES)

(21) Appl. No.: 17/253,381

(22) PCT Filed: Jun. 20, 2019
(86) PCT No.: PCT/EP2019/066383

§ 371 Date: Dec. 17, 2020

(30) Foreign Application Priority Data
Jun. 20, 2018 (EP) 18382446.5

(51) Int. Cl. C07D 401/14; C07D 471/04

(52) U.S. Cl. CPC C07D 401/14; C07D 471/04

(57) ABSTRACT

The present invention relates to O-GlcNAc hydrolase (OGA) inhibitors. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### FIELD OF THE INVENTION

The present invention relates to O-GlcNAc hydrolase (OGA) inhibitors, having the structure shown in Formula (I)

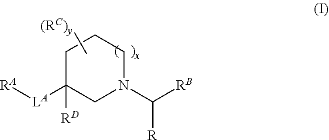

wherein the radicals are as defined in the specification. The invention is also directed to pharmaceutical compositions comprising such compounds, to processes for preparing such compounds and compositions, and to the use of such compounds and compositions for the prevention and treatment of disorders in which inhibition of OGA is beneficial, such as tauopathies, in particular Alzheimer's disease or progressive supranuclear palsy; and neurodegenerative diseases accompanied by a tau pathology, in particular amyotrophic lateral sclerosis or frontotemporal lobe dementia caused by C9ORF72 mutations.

### BACKGROUND OF THE INVENTION

O-GlcNAcylation is a reversible modification of proteins where N-acetyl-D-glucosamine residues are transferred to the hydroxyl groups of serine- and threonine residues yield O-GlcNAcylated proteins. More than 1000 of such target proteins have been identified both in the cytosol and nucleus of eukaryotes. The modification is thought to regulate a huge spectrum of cellular processes including transcription, cytoskeletal processes, cell cycle, proteasomal degradation, and receptor signalling.

O-GlcNAc transferase (OGT) and O-GlcNAc hydrolase (OGA) are the only two proteins described that add (OGT) or remove (OGA) O-GlcNAc from target proteins. OGA was initially purified in 1994 from spleen preparation and 1998 identified as antigen expressed by meningiomas and termed MGEA5, consists of 916 amino (102915 Dalton) as a monomer in the cytosolic compartment of cells. It is to be distinguished from ER- and Golgi-related glycosylation processes that are important for trafficking and secretion of proteins and different to OGA have an acidic pH optimum, whereas OGA display highest activity at neutral pH.

The OGA catalytic domain with its double aspartate catalytic center resides in the N-terminal part of the enzyme which is flanked by two flexible domains. The C-terminal part consists of a putative HAT (histone acetyl transferase domain) preceded by a stalk domain. It has yet still to be proven that the HAT-domain is catalytically active.

O-GlcNAcylated proteins as well as OGT and OGA themselves are particularly abundant in the brain and neurons suggesting this modification plays an important role in the central nervous system. Indeed, studies confirmed that O-GlcNAcylation represents a key regulatory mechanism contributing to neuronal communication, memory formation and neurodegenerative disease. Moreover, it has been shown that OGT is essential for embryogenesis in several animal models and ogt null mice are embryonic lethal. OGA is also indispensible for mammalian development. Two independent studies have shown that OGA homozygous null mice do not survive beyond 24-48 hours after birth. Oga deletion has led to defects in glycogen mobilization in pups and it caused genomic instability linked cell cycle arrest in MEFs derived from homozygous knockout embryos. The heterozygous animals survived to adulthood however they exhibited alterations in both transcription and metabolism.

It is known that perturbations in O-GlcNAc cycling impact chronic metabolic diseases such as diabetes, as well as cancer. Oga heterozygosity suppressed intestinal tumorigenesis in an Apc−/+ mouse cancer model and the Oga gene (MGEA5) is a documented human diabetes susceptibility locus.

In addition, O-GlcNAc-modifications have been identified on several proteins that are involved in the development and progression of neurodegenerative diseases and a correlation between variations of O-GlcNAc levels on the formation of neurofibrillary tangle (NFT) protein by Tau in Alzheimer's disease has been suggested. In addition, O-GlcNAcylation of alpha-synuclein in Parkinson's disease has been described.

In the central nervous system six splice variants of tau have been described. Tau is encoded on chromosome 17 and consists in its longest splice variant expressed in the central nervous system of 441 amino acids. These isoforms differ by two N-terminal inserts (exon 2 and 3) and exon 10 which lie within the microtubule binding domain. Exon 10 is of considerable interest in tauopathies as it harbours multiple mutations that render tau prone to aggregation as described below. Tau protein binds to and stabilizes the neuronal microtubule cytoskeleton which is important for regulation of the intracellular transport of organelles along the axonal compartments. Thus, tau plays an important role in the formation of axons and maintenance of their integrity. In addition, a role in the physiology of dendritic spines has been suggested as well.

Tau aggregation is either one of the underlying causes for a variety of so called tauopathies like PSP (progressive supranuclear palsy), Down's syndrome (DS), FTLD (frontotemporal lobe dementia), FTDP-17 (frontotemporal dementia with Parkinsonism-17), Pick's disease (PD), CBD (corticobasal degeneration), agryophilic grain disease (AGD), and AD (Alzheimer's disease). In addition, tau pathology accompanies additional neurodegenerative diseases like amyotrophic lateral sclerosis (ALS) or FTLD cause by C9ORF72 mutations. In these diseases, tau is post-translationally modified by excessive phosphorylation which is thought to detach tau from microtubules and makes it prone to aggregation. O-GlcNAcylation of tau regulates the extent of phosphorylation as serine or threonine residues carrying 0-GlcNAc-residues are not amenable to phosphorylation. This effectively renders tau less prone to detaching from microtubules and reduces aggregation into neurotoxic tangles which ultimately lead to neurotoxicity and neuronal cell death. This mechanism may also reduce the cell-to-cell spreading of tau-aggregates released by neurons via along interconnected circuits in the brain which has recently been discussed to accelerate pathology in tau-related dementias. Indeed, hyperphosphorylated tau isolated from brains of AD-patients showed significantly reduced O-GlcNAcylation levels.

An OGA inhibitor administered to JNPL3 tau transgenic mice successfully reduced NFT formation and neuronal loss without apparent adverse effects. This observation has been confirmed in another rodent model of tauopathy where the expression of mutant tau found in FTD can be induced (tg4510). Dosing of a small molecule inhibitor of OGA was efficacious in reducing the formation of tau-aggregation and attenuated the cortical atrophy and ventricle enlargement.

Moreover, the O-GlcNAcylation of the amyloid precursor protein (APP) favours processing via the non-amyloidogenic route to produce soluble APP fragment and avoid cleavage that results in the AD associated amyloid-beta (Aβ) formation.

Maintaining O-GlcNAcylation of tau by inhibition of OGA represents a potential approach to decrease tau-phosphorylation and tau-aggregation in neurodegenerative diseases mentioned above thereby attenuating or stopping the progression of neurodegenerative tauopathy-diseases.

WO2012/117219 (Summit Corp. plc., published 7 Sep. 2012) describes N-[[5-(hydroxymethyl)pyrrolidin-2-yl]methyl]alkylamide and N-alkyl-2-[5-(hydroxymethyl)pyrrolidin-2-yl]acetamide derivatives as OGA inhibitors; WO2016/0300443 (Asceneuron S. A., published 3 Mar. 2016), WO2017/144633 and WO2017/0114639 (Asceneuron S. A., published 31 Aug. 2017) disclose 1,4-disubstituted piperidines or piperazines as OGA inhibitors; WO2017/144637 (Asceneuron S. A, published 31 Aug. 2017) discloses more particular 4-substituted 1-[1-(1,3-benzodioxol-5-yl)ethyl]-piperazine; 1-[1-(2,3-dihydrobenzofuran-5-yl)ethyl]-; 1-[1-(2,3-dihydrobenzofuran-6-yl)ethyl]-; and 1-[1-(2,3-dihydro-1,4-benzodioxin-6-yl)ethyl]-piperazine derivatives as OGA inhibitors; WO2017/106254 (Merck Sharp & Dohme Corp.) describes substituted N-[5-[(4-methylene-1-piperidyl)methyl]thiazol-2-yl]acetamide compounds as OGA inhibitors.

There is still a need for OGA inhibitor compounds with an advantageous balance of properties, for example with improved potency, good bioavailability, pharmacokinetics, and brain penetration, and/or better toxicity profile. It is accordingly an object of the present invention to provide compounds that overcome at least some of these problems.

### SUMMARY OF THE INVENTION

The present invention is directed to compounds of Formula (I)

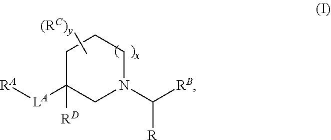

and the tautomers and the stereoisomeric forms thereof, wherein
R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl, each of which may be optionally substituted with 1, 2 or 3 substituents each independently selected from the group consisting of halo; cyano; C1-4alkyl optionally substituted with 1, 2, or 3 independently selected halo substituents; —C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents;
L^{A} is selected from the group consisting of a covalent bond, —O—, —CH₂—, —OCH₂—, —CH₂O—, —NH—, —N(CH₃)—, —NH—CH₂—, and —CH₂—NH—;
x represents 0 or 1;

### R is H or CH₃; and

R^{B} is an aromatic heterobicyclic radical selected from the group consisting of (b-1), (b-2), (b-3) and (b-4)

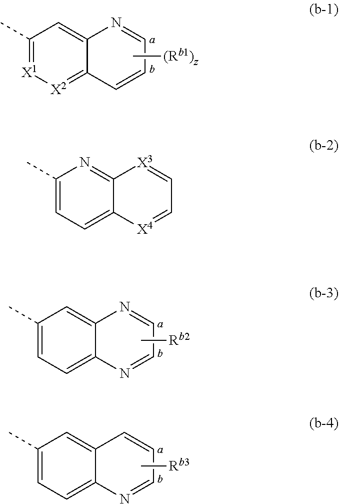

wherein
X¹ and X² are each independently selected from CH and N, with the proviso that at least one is CH;
z represents 0 or 1;
X³ and X⁴ are each independently selected from CH and N, with the proviso that X³ is CH when X⁴ is N, and X³ is N when X⁴ is CH;
R^{b1}, R^{b2}, and R^{b3} are each independently selected from the group consisting of methyl, hydroxy, and halo;
a and b represent the optional position of attachment for R^{b1}, R^{b2}, or R^{b3};
R^{C} is selected from the group consisting of fluoro, methyl, hydroxy, methoxy, trifluoromethyl, and difluoromethyl;
R^{D} is selected from the group consisting of hydrogen, fluoro, methyl, hydroxy, methoxy, trifluoromethyl, and difluoromethyl; and
y represents 0, 1 or 2;
with the provisos that
a) R^{C} is not hydroxy or methoxy when present at the carbon atom adjacent to the nitrogen atom of the piperidinediyl or pyrrolidinediyl ring;
b) R^{C} or R^{D} cannot be selected simultaneously from hydroxy or methoxy when R^{C} is present at the carbon atom adjacent to C—R^{P}; and
c) R^{D} is not hydroxy or methoxy when L^{A} is —O—, —OCH₂—, —CH₂O—, —NH—, —N(CH₃)—, —NHCH₂— or —CH₂NH—;
   and the pharmaceutically acceptable salts and the solvates thereof.

Illustrative of the invention is a pharmaceutical composition comprising a pharmaceutically acceptable carrier and any of the compounds described above. An illustration of the invention is a pharmaceutical composition made by mixing any of the compounds described above and a pharmaceutically acceptable carrier. Illustrating the invention is a process for making a pharmaceutical composition comprising mixing any of the compounds described above and a pharmaceutically acceptable carrier.

Exemplifying the invention are methods of preventing or treating a disorder mediated by the inhibition of O-GlcNAc hydrolase (OGA), comprising administering to a subject in need thereof a therapeutically effective amount of any of the compounds or pharmaceutical compositions described above.

Further exemplifying the invention are methods of inhibiting OGA, comprising administering to a subject in need thereof a prophylactically or a therapeutically effective amount of any of the compounds or pharmaceutical compositions described above.

An example of the invention is a method of preventing or treating a disorder selected from a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of any of the compounds or pharmaceutical compositions described above.

Another example of the invention is any of the compounds described above for use in preventing or treating a tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, in a subject in need thereof.

### DETAILED DESCRIPTION OF THE INVENTION

The present invention is directed to compounds of Formula (I), as defined herein before, and pharmaceutically acceptable addition salts and solvates thereof. The compounds of Formula (I) are inhibitors of O-GlcNAc hydrolase (OGA) and may be useful in the prevention or treatment of tauopathies, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or maybe useful in the prevention or treatment of neurodegenerative diseases accompanied by a tau pathology, in particular a neurodegenerative disease selected from amyotrophic lateral sclerosis or frontotemporal lobe dementia caused by C9ORF72 mutations.

In a particular embodiment, the invention is directed to compounds of Formula (I) as defined hereinbefore, and the tautomers and the stereoisomeric forms thereof, wherein R^{B} is (b-1), (b-2) or (b-3), wherein

X¹ and X² are both CH; or X¹ is CH and X² is N; or X¹ is N and X² is CH;
z is 0 or 1;
R^{b1} is CH₃;
X³ is N and X⁴ is CH; and

### R^{b2} is OH.

In a further particular embodiment, the invention is directed to compounds of Formula (I) as defined hereinbefore, and the tautomers and the stereoisomeric forms thereof, wherein R^{B} is (b-1); wherein

X¹ and X² are both CH; or X¹ is CH and X² is N; or X¹ is N and X² is CH;
z is 0 or 1; and
R^{b1} is CH₃, bound at position a.

In a further particular embodiment, the invention is directed to compounds of Formula (I) as defined hereinbefore, and the tautomers and the stereoisomeric forms thereof, wherein R^{B} is (b-2); wherein X³ is N and X⁴ is CH.

In a further particular embodiment, the invention is directed to compounds of Formula (I) as defined hereinbefore, and the tautomers and the stereoisomeric forms thereof, wherein R^{B} is (b-3); wherein R^{b2} is bound at position b.

In a particular embodiment, the invention is directed to compounds of Formula (I) as defined hereinbefore, and the tautomers and the stereoisomeric forms thereof, wherein y is 0 and R^{D} is hydrogen.

In an additional embodiment, the invention is directed to compounds of Formula (I) as defined hereinbefore, and the tautomers and the stereoisomeric forms thereof, wherein R^{A} is pyridin-4-yl; optionally substituted with 1 or 2 substituents, each independently selected from the group consisting of halo; C1-4alkyl optionally substituted with 1, 2, or 3 independently selected halo substituents; and C1-4alkyloxy optionally substituted with 1, 2, or 3 independently selected halo substituents.

In a further embodiment, the invention is directed to compounds of Formula (I) as defined hereinbefore, and the tautomers and the stereoisomeric forms thereof, wherein R^{A} is pyridin-4-yl; optionally substituted with 1 or 2 substituents, each independently selected from the group consisting of C1-4alkyl optionally substituted with 1, 2, or 3 independently selected halo substituents; and C1-4alkyloxy optionally substituted with 1, 2, or 3 independently selected halo substituents. More in particular, R^{A} is pyridin-4-yl substituted with 1 or 2 independently selected C1-4alkyl substituents.

In a further embodiment, the invention is directed to compounds of Formula (I) as defined hereinbefore, and the tautomers and the stereoisomeric forms thereof, wherein L^{A} is selected from the group consisting of —O—, —CH₂—, —OCH₂—, —CH₂O—, —NH—, —N(CH₃)—, —NH—CH₂—, and —CH₂—NH—.

In a further embodiment, the invention is directed to compounds of Formula (I) as defined hereinbefore, and the tautomers and the stereoisomeric forms thereof, wherein L^{A} is —CH₂—.

In a further embodiment, the invention is directed to compounds of Formula (I), as referred to herein, and the tautomers and the stereoisomeric forms thereof, wherein R^{B} is selected from the group consisting of

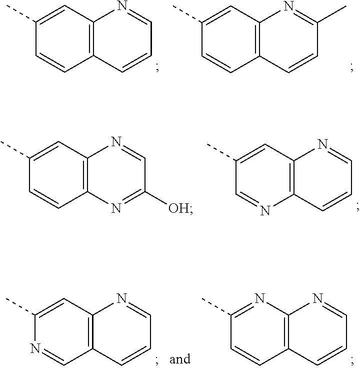

and the pharmaceutically acceptable salts and the solvates thereof.

In a further embodiment, the invention is directed to compounds of Formula (I), as referred to herein, and the tautomers and the stereoisomeric forms thereof, wherein R^{B} is

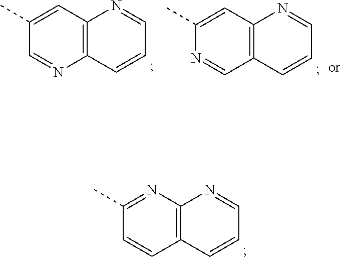

and the pharmaceutically acceptable salts and the solvates thereof.

In a further embodiment, the invention is directed to compounds of Formula (I), as referred to herein, and the tautomers and the stereoisomeric forms thereof, wherein R^{A} is

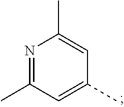

and the pharmaceutically acceptable salts and the solvates thereof.

### Definitions

“Halo” shall denote fluoro, chloro and bromo; “C₁₋₄alkyl” shall denote a straight or branched saturated alkyl group having 1, 2, 3 or 4 carbon atoms, respectively e.g. methyl, ethyl, 1-propyl, 2-propyl, butyl, 1-methyl-propyl, 2-methyl-1-propyl, 1,1-dimethylethyl, and the like; “C₁₋₄alkyloxy” shall denote an ether radical wherein C1-4alkyl is as defined before. When reference is made to L^{A}, the definition is to be read from left to right, with the left part of the linker bound to R^{A} and the right part of the linker bound to the pyrrolidinediyl or piperidinediyl ring. Thus, when L^{A} is, for example, —O—CH₂—, then R^{A}-L^{A}- is R^{A}—O—CH₂—. When R^{C} is present more than once, where possible, it may be bound at the same carbon atom of the pyrrolidinediyl or piperidinediyl ring, and each instance may be different.

In general, whenever the term “substituted” is used in the present invention, it is meant, unless otherwise indicated or is clear from the context, to indicate that one or more hydrogens, in particular 1 to 3 hydrogens, preferably 1 or 2 hydrogens, more preferably 1 hydrogen, on the atom or radical indicated in the expression using “substituted” are replaced with a selection of substituents from the indicated group, provided that the normal valency is not exceeded, and that the substitution results in a chemically stable compound, i.e. a compound that is sufficiently robust to survive isolation to a useful degree of purity from a reaction mixture, and formulation into a therapeutic agent.

The term “subject” as used herein, refers to an animal, preferably a mammal, most preferably a human, who is or has been the object of treatment, observation or experiment. As used herein, the term “subject” therefore encompasses patients, as well as asymptomatic or presymptomatic individuals at risk of developing a disease or condition as defined herein.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. The term “prophylactically effective amount” as used herein, means that amount of active compound or pharmaceutical agent that substantially reduces the potential for onset of the disease or disorder being prevented.

As used herein, the term “composition” is intended to encompass a product comprising the specified ingredients in the specified amounts, as well as any product which results, directly or indirectly, from combinations of the specified ingredients in the specified amounts.

Hereinbefore and hereinafter, the term “compound of Formula (I)” is meant to include the addition salts, the solvates and the stereoisomers thereof.

The terms “stereoisomers” or “stereochemically isomeric forms” hereinbefore or hereinafter are used interchangeably.

The invention includes all stereoisomers of the compound of Formula (I) either as a pure stereoisomer or as a mixture of two or more stereoisomers.

Enantiomers are stereoisomers that are non-superimposable mirror images of each other. A 1:1 mixture of a pair of enantiomers is a racemate or racemic mixture.

Diastereomers (or diastereoisomers) are stereoisomers that are not enantiomers, i.e. they are not related as mirror images. If a compound contains a double bond, the substituents may be in the E or the Z configuration. If a compound contains a disubstituted cycloalkyl group, the substituents may be in the cis or trans configuration. Therefore, the invention includes enantiomers, diastereomers, racemates, E isomers, Z isomers, cis isomers, trans isomers and mixtures thereof.

The absolute configuration is specified according to the Cahn-Ingold-Prelog system. The configuration at an asymmetric atom is specified by either R or S. Resolved compounds whose absolute configuration is not known can be designated by (+) or (−) depending on the direction in which they rotate plane polarized light.

When a specific stereoisomer is identified, this means that said stereoisomer is substantially free, i.e. associated with less than 50%, preferably less than 20%, more preferably less than 10%, even more preferably less than 5%, in particular less than 2% and most preferably less than 1%, of the other isomers. Thus, when a compound of formula (I) is for instance specified as (R), this means that the compound is substantially free of the (S) isomer; when a compound of formula (I) is for instance specified as E, this means that the compound is substantially free of the Z isomer; when a compound of formula (I) is for instance specified as cis, this means that the compound is substantially free of the trans isomer.

For use in medicine, the addition salts of the compounds of this invention refer to non-toxic “pharmaceutically acceptable addition salts”. Other salts may, however, be useful in the preparation of compounds according to this invention or of their pharmaceutically acceptable addition salts. Suitable pharmaceutically acceptable addition salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable addition salts thereof may include alkali metal salts, e.g., sodium or potassium salts; alkaline earth metal salts, e.g., calcium or magnesium salts; and salts formed with suitable organic ligands, e.g., quaternary ammonium salts.

Representative acids which may be used in the preparation of pharmaceutically acceptable addition salts include, but are not limited to, the following: acetic acid, 2,2-dichloroactic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, capric acid, caproic acid, caprylic acid, cinnamic acid, citric acid, cyclamic acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, beta-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, trifluoromethylsulfonic acid, and undecylenic acid. Representative bases which may be used in the preparation of pharmaceutically acceptable addition salts include, but are not limited to, the following: ammonia, L-arginine, benethamine, benzathine, calcium hydroxide, choline, dimethylethanol-amine, diethanolamine, diethylamine, 2-(diethylamino)-ethanol, ethanolamine, ethylene-diamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The names of compounds were generated according to the nomenclature rules agreed upon by the Chemical Abstracts Service (CAS) or according to the nomenclature rules agreed upon by the International Union of Pure and Applied Chemistry (IUPAC).

### Preparation of the Final Compounds

The compounds according to the invention can generally be prepared by a succession of steps, each of which is known to the skilled person. In particular, the compounds can be prepared according to the following synthesis methods.

The compounds of Formula (I) may be synthesized in the form of racemic mixtures of enantiomers which can be separated from one another following art-known resolution procedures. The racemic compounds of Formula (I) may be converted into the corresponding diastereomeric salt forms by reaction with a suitable chiral acid.

Said diastereomeric salt forms are subsequently separated, for example, by selective or fractional crystallization and the enantiomers are liberated therefrom by alkali. An alternative manner of separating the enantiomeric forms of the compounds of Formula (I) involves liquid chromatography using a chiral stationary phase. Said pure stereochemically isomeric forms may also be derived from the corresponding pure stereochemically isomeric forms of the appropriate starting materials, provided that the reaction occurs stereospecifically.

### Experimental Procedure 1

The final compounds of Formula (I-a) can be prepared by reacting an intermediate compound of Formula (II) with a compound of Formula (XV) according to reaction scheme (1). The reaction is performed in a suitable reaction-inert solvent, such as, for example, dichloromethane, a metal hydride, such as, for example sodium triacetoxyborohydride, sodium cyanoborohydride or sodium borohydride and may require the presence of a suitable base, such as, for example, triethylamine, and/or a Lewis acid, such as, for example titanium tetraisopropoxide or titanium tetrachloride, under thermal conditions, such as, 0° C. or room temperature, or 140° C., for example for 1 hour or 24 hours. In reaction scheme (1) all variables are defined as in Formula (I).

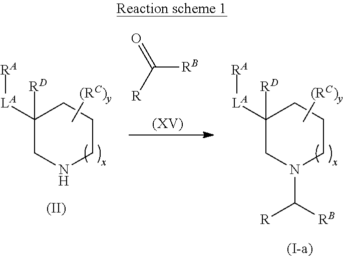

### Experimental Procedure 2

Additionally final compounds of Formula (I-a) can be prepared by reacting an intermediate compound of Formula (II) with a compound of Formula (XVI) according to reaction scheme (2). The reaction is performed in a suitable reaction-inert solvent, such as, for example, acetonitrile, a suitable base, such as, for example, triethylamine or diisopropylethylamine, under thermal conditions, such as, 0° C. or room temperature, or 75° C., for example for 1 hour or 24 hours. In reaction scheme (2) all variables are defined as in Formula (I), and wherein halo is chloro, bromo or iodo.

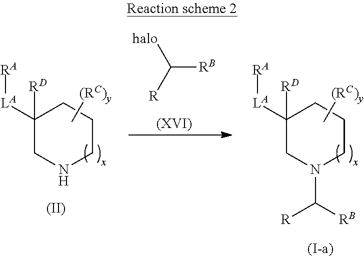

### Experimental Procedure 3

Additionally final compounds of Formula (I), wherein R═CH₃, herein referred to as (I-b), can be prepared by reacting an intermediate compound of Formula (II) with a compound of Formula (XVII) followed by reaction of the formed imine derivative with and intermediate compound of Formula (XVIII) according to reaction scheme (3). The reaction is performed in a suitable reaction-inert solvent, such as, for example, anhydrous dichloromethane, a Lewis acid, such as, for example titanium tetraisopropoxide or titanium tetrachloride, under thermal conditions, such as, 0° C. or room temperature, for example for 1 hour or 24 hours. In reaction scheme (3) all variables are defined as in Formula (I), and wherein halo is chloro, bromo or iodo.

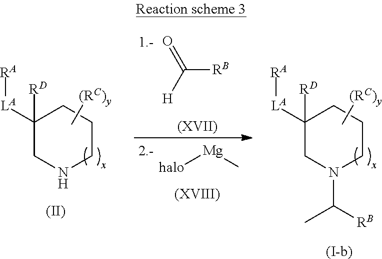

### Experimental Procedure 4

Additionally final compounds of Formula (I), wherein L^{A}=NHCH₂, herein referred to as (I-c), can be prepared by reacting an intermediate compound of Formula (III) with a compound of Formula (V) according to reaction scheme (4). The reaction is performed in the presence of a palladium catalyst, such as, for example tris(dibenzylideneacetone)dipalladium(0), a ligand, such as, for example 2-dicyclohexylphosphino-2′-(N,N-dimethylamino)biphenyl, a base, such as, for example sodium tert-butoxide, a suitable reaction-inert solvent, such as, for example, anhydrous 1,4-dioxane, under thermal conditions, such as, 100° C., for example for 4 hour or 24 hours. In reaction scheme (4) all variables are defined as in Formula (I), and wherein halo is chloro, bromo or iodo.

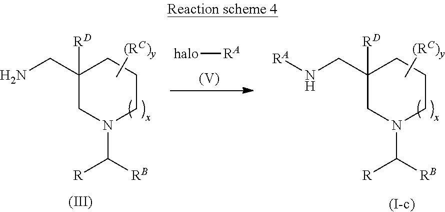

### Experimental Procedure 5

Intermediate compounds of Formula (II) can be prepared cleaving a protecting group in an intermediate compound of Formula (IV) according to reaction scheme (5). In reaction scheme (5) all variables are defined as in Formula (I), and PG is a suitable protecting group of the nitrogen function such as, for example, tert-butoxycarbonyl (Boc), ethoxycarbonyl, benzyl, benzyloxycarbonyl (Cbz). Suitable methods for removing such protecting groups are widely known to the person skilled in the art and comprise but are not limited to: Boc deprotection: treatment with a protic acid, such as, for example, trifluoroacetic acid, in a reaction inert solvent, such as, for example, dichloromethane; ethoxycarbonyl deprotection: treatment with a strong base, such as, for example, sodium hydroxide, in a reaction inert solvent such as for example wet tetrahydrofuran; benzyl deprotection: catalytic hydrogenation in the presence of a suitable catalyst, such as, for example, palladium on carbon, in a reaction inert solvent, such as, for example, ethanol; benzyloxycarbonyl deprotection: catalytic hydrogenation in the presence of a suitable catalyst, such as, for example, palladium on carbon, in a reaction inert solvent, such as, for example, ethanol.

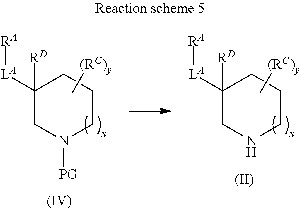

### Experimental Procedure 6

Intermediate compounds of Formula (IV-a) can be prepared by “Negishi coupling” reaction of a halo compound of Formula (V) with an organozinc compound of Formula (VI) according to reaction scheme (6). The reaction is performed in a suitable reaction-inert solvent, such as, for example, tetrahydrofuran, and a suitable catalyst, such as, for example, Pd(OAc)₂, a suitable ligand for the transition metal, such as, for example, 2-dicyclohexylphosphino-2′,6′-diisopropoxybiphenyl [CAS: 787618-22-8], under thermal conditions, such as, for example, room temperature, for example for 1 hour. In reaction scheme (6) all variables are defined as in Formula (I), L^{A} is a bond or CH₂ and halo is preferably bromo or iodo. PG is defined as in Formula (IV).

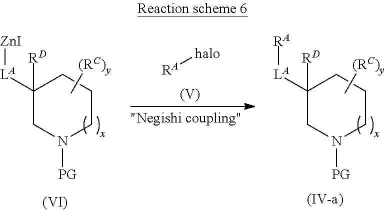

### Experimental Procedure 7

Intermediate compounds of Formula (VI) can be prepared by reaction of a halo compound of Formula (VII) with zinc according to reaction scheme (7). The reaction is performed in a suitable reaction-inert solvent, such as, for example, tetrahydrofuran, and a suitable salt, such as, for example, lithium chloride, under thermal conditions, such as, for example, 40° C., for example in a continuous-flow reactor. In reaction scheme (7) all variables are defined as in Formula (I), L^{A} is a bond or CH₂ and halo is preferably iodo. PG is defined as in Formula (IV).

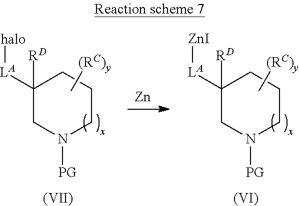

### Experimental Procedure 8

Intermediate compounds of Formula (IV), wherein L^{A} is a covalent bond and R^{D} is H, herein referred to as (IV-b), can be prepared by hydrogenation reaction of an alkene compound of Formula (VIII) according to reaction scheme (8). The reaction is performed in a suitable reaction-inert solvent, such as, for example, methanol, and a suitable catalyst, such as, for example, palladium on carbon, and hydrogen, under thermal conditions, such as, for example, room temperature, for example for 3 hours. In reaction scheme (8) all variables are defined as in Formula (I) and PG is defined as in Formula (IV).

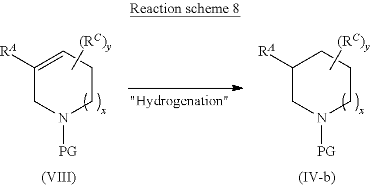

### Experimental Procedure 9

Intermediate compounds of Formula (VIII) can be prepared by “Suzuki coupling” reaction of an alkene compound of Formula (IX) and a halo derivative of Formula (V) according to reaction scheme (9). The reaction is performed in a suitable reaction-inert solvent, such as, for example, 1,4-dioxane, and a suitable catalyst, such as, for example, tetrakis(triphenylphosphine)palladium(0), a suitable base, such as, for example, NaHCO₃ (aq. sat. soltn.), under thermal conditions, such as, for example, 130° C., for example for 30 min under microwave irradiation. In reaction scheme (9) all variables are defined as in Formula (I), halo is preferably bromo or iodo, and PG is defined as in Formula (IV).

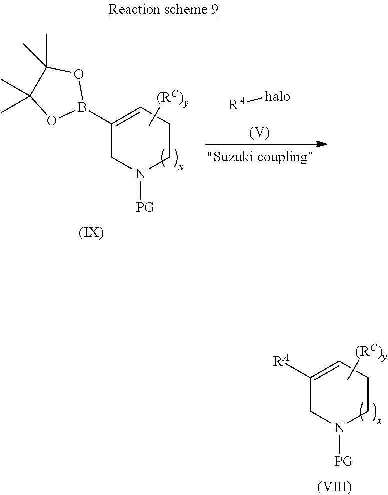

### Experimental Procedure 10

Intermediate compounds of Formula (IV-c) can be prepared by reaction of a hydroxy compound of Formula (X) and a halo derivative of Formula (V) according to reaction scheme (10). The reaction is performed in a suitable reaction-inert solvent, such as, for example, dimethylformamide or dimethylsulfoxide, and a suitable base, such as, sodium hydride or potassium tert-butoxide, under thermal conditions, such as, for example, 50° C., for example for 48 hour. In reaction scheme (10) all variables are defined as in Formula (I), L^{A′} is a bond or CH₂ and halo is preferably chloro, bromo or fluoro. PG is defined as in Formula (IV).

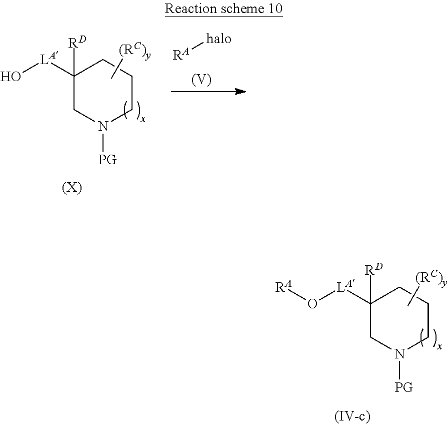

### Experimental Procedure 11

Alternatively intermediate compounds of Formula (IV), wherein L^{A} is O or OCH₂, herein referred to as (IV-c), can be prepared by “Mitsunobu reaction” of a hydroxy compound of Formula (X) and a hydroxy derivative of Formula (XI) according to reaction scheme (11). The reaction is performed in a suitable reaction-inert solvent, such as, for example, toluene, a phosphine, such as, triphenylphosphine, a suitable coupling agent, such as, for example DIAD (CAS: 2446-83-5), under thermal conditions, such as, for example, 70° C., for example for 17 hour. In reaction scheme (11) all variables are defined as in Formula (I), L^{A′} is a bond or CH₂ and halo is preferably chloro, bromo or fluoro. PG is defined as in Formula (IV).

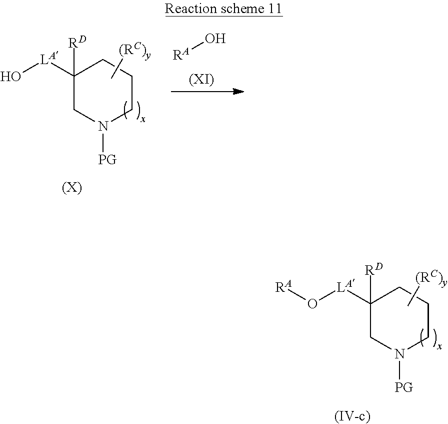

### Experimental Procedure 12

Intermediate compounds of Formula (III) can be prepared cleaving the protecting group in an intermediate compound of Formula (XI) according to reaction scheme (12). The reaction is performed in the presence of hydrazine hydrate in a suitable reaction-inert solvent, such as, for example, ethanol, under thermal conditions, such as, for example, 80° C., for example for 2 hour. In reaction scheme (12) all variables are defined as in Formula (I).

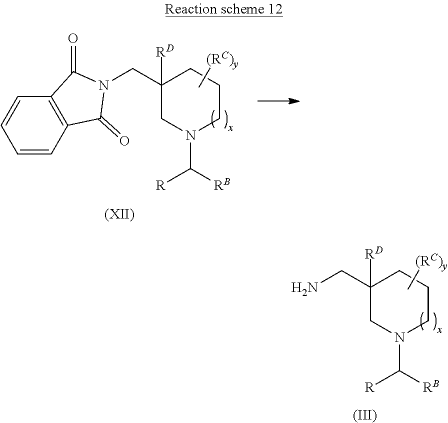

### Experimental Procedure 13

Intermediate compounds of Formula (XII) can be prepared by reacting an intermediate compound of Formula (XIII) with phtalimide according to reaction scheme (13). The reaction is performed in the presence of a phosphine, such as, for example triphenylphosphine, a suitable coupling agent, such as, for example diisopropyl azodicarboxylate in a suitable reaction-inert solvent, such as, for example, dry tetrahydrofuran, under thermal conditions, such as, for example, room temperature, for example for 24 hour. In reaction scheme (13) all variables are defined as in Formula (I).

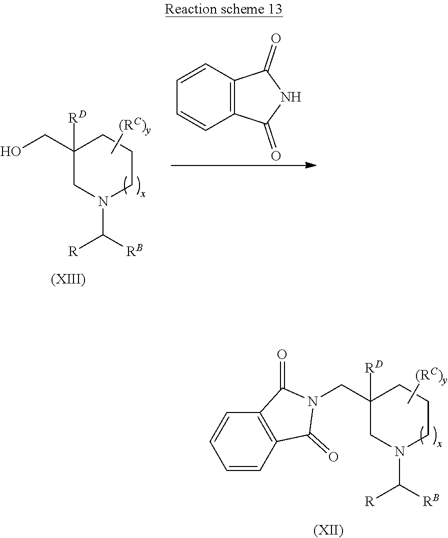

### Experimental Procedure 14

Intermediate compounds of Formula (XIII) can be prepared by deprotecting the alcohol group in an intermediate compound of Formula (XIV) according to reaction scheme (14). The reaction is performed in the presence of a fluoride source, such as, for example tetrabutylammonium fluoride, in a suitable reaction-inert solvent, such as, for example, dry tetrahydrofuran, under thermal conditions, such as, for example, room temperature, for example for 16 hour. In reaction scheme (13) all variables are defined as in Formula (I) and PG¹ is selected from the group consisting of trimethylsilyl, tert-butyldimethylsilyl, triisopropylsilyl or tert-butyldiphenylsilyl.

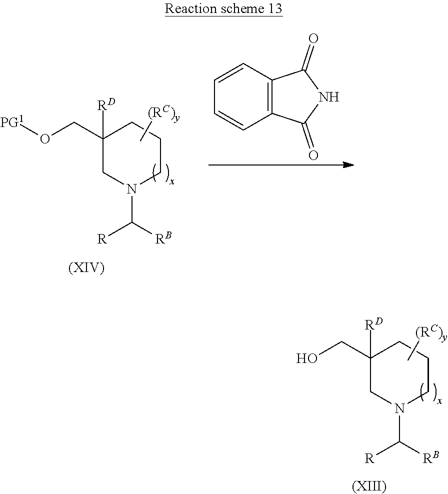

Intermediates of Formulae (V), (VII), (IX), (XV), (XVI), (XVII) and (XVIII) are commercially available or can be prepared by know procedures to those skilled in the art.

### Pharmacology

The compounds of the present invention and the pharmaceutically acceptable compositions thereof inhibit O-GlcNAc hydrolase (OGA) and therefore may be useful in the treatment or prevention of diseases involving tau pathology, also known as tauopathies, and diseases with tau inclusions. Such diseases include, but are not limited to Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Straussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

As used herein, the term “treatment” is intended to refer to all processes, wherein there may be a slowing, interrupting, arresting or stopping of the progression of a disease or an alleviation of symptoms, but does not necessarily indicate a total elimination of all symptoms. As used herein, the term “prevention” is intended to refer to all processes, wherein there may be a slowing, interrupting, arresting or stopping of the onset of a disease.

The invention also relates to a compound according to the general Formula (I), a stereoisomeric form thereof or a pharmaceutically acceptable acid or base addition salt thereof, for use in the treatment or prevention of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Straussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

The invention also relates to a compound according to the general Formula (I), a stereoisomeric form thereof or a pharmaceutically acceptable acid or base addition salt thereof, for use in the treatment, prevention, amelioration, control or reduction of the risk of diseases or conditions selected from the group consisting of Alzheimer's disease, amyotrophic lateral sclerosis and parkinsonism-dementia complex, argyrophilic grain disease, chronic traumatic encephalopathy, corticobasal degeneration, diffuse neurofibrillary tangles with calcification, Down's syndrome, Familial British dementia, Familial Danish dementia, Frontotemporal dementia and parkinsonism linked to chromosome 17 (caused by MAPT mutations), Frontotemporal lobar degeneration (some cases caused by C9ORF72 mutations), Gerstmann-Straussler-Scheinker disease, Guadeloupean parkinsonism, myotonic dystrophy, neurodegeneration with brain iron accumulation, Niemann-Pick disease, type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, SLC9A6-related mental retardation, subacute sclerosing panencephalitis, tangle-only dementia, and white matter tauopathy with globular glial inclusions.

In particular, the diseases or conditions may in particular be selected from a tauopathy, more in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or the diseases or conditions may in particular be neurodegenerative diseases accompanied by a tau pathology, more in particular a neurodegenerative disease selected from amyotrophic lateral sclerosis or frontotemporal lobe dementia caused by C9ORF72 mutations.

Preclinical states in Alzheimer's and tauopathy diseases: In recent years the United States (US) National Institute for Aging and the International Working Group have proposed guidelines to better define the preclinical (asymptomatic) stages of AD (Dubois B, et al. Lancet Neurol. 2014; 13:614-629; Sperling, R A, et al. Alzheimers Dement. 2011; 7:280-292). Hypothetical models postulate that Aβ accumulation and tau-aggregation begins many years before the onset of overt clinical impairment. The key risk factors for elevated amyloid accumulation, tau-aggregation and development of AD are age (ie, 65 years or older), APOE genotype, and family history. Approximately one third of clinically normal older individuals over 75 years of age demonstrate evidence of Aβ or tau accumulation on PET amyloid and tau imaging studies, the latter being less advanced currently. In addition, reduced Abeta-levels in CSF measurements are observed, whereas levels of non-modified as well as phosphorylated tau are elevated in CSF. Similar findings are seen in large autopsy studies and it has been shown that tau aggregates are detected in the brain as early as 20 years of age and younger. Amyloid-positive (Aβ+) clinically normal individuals consistently demonstrate evidence of an “AD-like endophenotype” on other biomarkers, including disrupted functional network activity in both functional magnetic resonance imaging (MRI) and resting state connectivity, fluorodeoxyglucose ¹⁸F (FDG) hypometabolism, cortical thinning, and accelerated rates of atrophy. Accumulating longitudinal data also strongly suggests that Aβ+ clinically normal individuals are at increased risk for cognitive decline and progression to mild cognitive impairment (MCI) and AD dementia. The Alzheimer's scientific community is of the consensus that these Aβ+ clinically normal individuals represent an early stage in the continuum of AD pathology. Thus, it has been argued that intervention with a therapeutic agent that decreases Aβ production or the aggregation of tau is likely to be more effective if started at a disease stage before widespread neurodegeneration has occurred. A number of pharmaceutical companies are currently testing BACE inhibition in prodromal AD.

Thanks to evolving biomarker research, it is now possible to identify Alzheimer's disease at a preclinical stage before the occurrence of the first symptoms. All the different issues relating to preclinical Alzheimer's disease such as, definitions and lexicon, the limits, the natural history, the markers of progression and the ethical consequences of detecting the disease at the asymptomatic stage, are reviewed in Alzheimer's & Dementia 12 (2016) 292-323.

Two categories of individuals may be recognized in preclinical Alzheimer's disease or tauopathies. Cognitively normal individuals with amyloid beta or tau aggregation evident on PET scans, or changes in CSF Abeta, tau and phospho-tau are defined as being in an “asymptomatic at risk state for Alzheimer's disease (AR-AD)” or in a “asymptomatic state of tauopathy”. Individuals with a fully penetrant dominant autosomal mutation for familial Alzheimer's disease are said to have “presymptomatic Alzheimer's disease”. Dominant autosomal mutations within the tau-protein have been described for multiple forms of tauopathies as well.

Thus, in an embodiment, the invention also relates to a compound according to the general Formula (I), a stereoisomeric form thereof or a pharmaceutically acceptable acid or base addition salt thereof, for use in control or reduction of the risk of preclinical Alzheimer's disease, prodromal Alzheimer's disease, or tau-related neurodegeneration as observed in different forms of tauopathies.

As already mentioned hereinabove, the term “treatment” does not necessarily indicate a total elimination of all symptoms, but may also refer to symptomatic treatment in any of the disorders mentioned above. In view of the utility of the compound of Formula (I), there is provided a method of treating subjects such as warm-blooded animals, including humans, suffering from or a method of preventing subjects such as warm-blooded animals, including humans, suffering from any one of the diseases mentioned hereinbefore.

Said methods comprise the administration, i.e. the systemic or topical administration, preferably oral administration, of a prophylactically or a therapeutically effective amount of a compound of Formula (I), a stereoisomeric form thereof, a pharmaceutically acceptable addition salt or solvate thereof, to a subject such as a warm-blooded animal, including a human.

Therefore, the invention also relates to a method for the prevention and/or treatment of any of the diseases mentioned hereinbefore comprising administering a prophylactically or a therapeutically effective amount of a compound according to the invention to a subject in need thereof.

The invention also relates to a method for modulating O-GlcNAc hydrolase (OGA) activity, comprising administering to a subject in need thereof, a prophylactically or a therapeutically effective amount of a compound according to the invention and as defined in the claims or a pharmaceutical composition according to the invention and as defined in the claims.

A method of treatment may also include administering the active ingredient on a regimen of between one and four intakes per day. In these methods of treatment the compounds according to the invention are preferably formulated prior to administration. As described herein below, suitable pharmaceutical formulations are prepared by known procedures using well known and readily available ingredients.

The compounds of the present invention, that can be suitable to treat or prevent any of the disorders mentioned above or the symptoms thereof, may be administered alone or in combination with one or more additional therapeutic agents. Combination therapy includes administration of a single pharmaceutical dosage formulation which contains a compound of Formula (I) and one or more additional therapeutic agents, as well as administration of the compound of Formula (I) and each additional therapeutic agent in its own separate pharmaceutical dosage formulation. For example, a compound of Formula (I) and a therapeutic agent may be administered to the patient together in a single oral dosage composition such as a tablet or capsule, or each agent may be administered in separate oral dosage formulations.

A skilled person will be familiar with alternative nomenclatures, nosologies, and classification systems for the diseases or conditions referred to herein. For example, the fifth edition of the Diagnostic & Statistical Manual of Mental Disorders (DSM-5™) of the American Psychiatric Association utilizes terms such as neurocognitive disorders (NCDs) (both major and mild), in particular, neurocognitive disorders due to Alzheimer's disease. Such terms may be used as an alternative nomenclature for some of the diseases or conditions referred to herein by the skilled person.

### Pharmaceutical Compositions

The present invention also provides compositions for preventing or treating diseases in which inhibition of O-GlcNAc hydrolase (OGA) is beneficial, such as Alzheimer's disease, progressive supranuclear palsy, Down's syndrome, frontotemporal lobe dementia, frontotemporal dementia with Parkinsonism-17, Pick's disease, corticobasal degeneration, agryophilic grain disease, amyotrophic lateral sclerosis or frontotemporal lobe dementia caused by C9ORF72 mutations, said compositions comprising a therapeutically effective amount of a compound according to formula (I) and a pharmaceutically acceptable carrier or diluent.

While it is possible for the active ingredient to be administered alone, it is preferable to present it as a pharmaceutical composition. Accordingly, the present invention further provides a pharmaceutical composition comprising a compound according to the present invention, together with a pharmaceutically acceptable carrier or diluent. The carrier or diluent must be “acceptable” in the sense of being compatible with the other ingredients of the composition and not deleterious to the recipients thereof.

The pharmaceutical compositions of this invention may be prepared by any methods well known in the art of pharmacy. A therapeutically effective amount of the particular compound, in base form or addition salt form, as the active ingredient is combined in intimate admixture with a pharmaceutically acceptable carrier, which may take a wide variety of forms depending on the form of preparation desired for administration. These pharmaceutical compositions are desirably in unitary dosage form suitable, preferably, for systemic administration such as oral, percutaneous or parenteral administration; or topical administration such as via inhalation, a nose spray, eye drops or via a cream, gel, shampoo or the like. For example, in preparing the compositions in oral dosage form, any of the usual pharmaceutical media may be employed, such as, for example, water, glycols, oils, alcohols and the like in the case of oral liquid preparations such as suspensions, syrups, elixirs and solutions; or solid carriers such as starches, sugars, kaolin, lubricants, binders, disintegrating agents and the like in the case of powders, pills, capsules and tablets. Because of their ease in administration, tablets and capsules represent the most advantageous oral dosage unit form, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. In the compositions suitable for percutaneous administration, the carrier optionally comprises a penetration enhancing agent and/or a suitable wettable agent, optionally combined with suitable additives of any nature in minor proportions, which additives do not cause any significant deleterious effects on the skin. Said additives may facilitate the administration to the skin and/or may be helpful for preparing the desired compositions. These compositions may be administered in various ways, e.g., as a transdermal patch, as a spot-on or as an ointment.

It is especially advantageous to formulate the aforementioned pharmaceutical compositions in dosage unit form for ease of administration and uniformity of dosage. Dosage unit form as used in the specification and claims herein refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

The exact dosage and frequency of administration depends on the particular compound of Formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

The present compounds can be used for systemic administration such as oral, percutaneous or parenteral administration; or topical administration such as via inhalation, a nose spray, eye drops or via a cream, gel, shampoo or the like. The compounds are preferably orally administered. The exact dosage and frequency of administration depends on the particular compound according to Formula (I) used, the particular condition being treated, the severity of the condition being treated, the age, weight, sex, extent of disorder and general physical condition of the particular patient as well as other medication the individual may be taking, as is well known to those skilled in the art. Furthermore, it is evident that said effective daily amount may be lowered or increased depending on the response of the treated subject and/or depending on the evaluation of the physician prescribing the compounds of the instant invention.

The amount of a compound of Formula (I) that can be combined with a carrier material to produce a single dosage form will vary depending upon the disease treated, the mammalian species, and the particular mode of administration. However, as a general guide, suitable unit doses for the compounds of the present invention can, for example, preferably contain between 0.1 mg to about 1000 mg of the active compound. A preferred unit dose is between 1 mg to about 500 mg. A more preferred unit dose is between 1 mg to about 300 mg. Even more preferred unit dose is between 1 mg to about 100 mg. Such unit doses can be administered more than once a day, for example, 2, 3, 4, 5 or 6 times a day, but preferably 1 or 2 times per day, so that the total dosage for a 70 kg adult is in the range of 0.001 to about 15 mg per kg weight of subject per administration. A preferred dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

The invention also provides a kit comprising a compound according to the invention, prescribing information also known as “leaflet”, a blister package or bottle, and a container. Furthermore, the invention provides a kit comprising a pharmaceutical composition according to the invention, prescribing information also known as “leaflet”, a blister package or bottle, and a container. The prescribing information preferably includes advice or instructions to a patient regarding the administration of the compound or the pharmaceutical composition according to the invention. In particular, the prescribing information includes advice or instruction to a patient regarding the administration of said compound or pharmaceutical composition according to the invention, on how the compound or the pharmaceutical composition according to the invention is to be used, for the prevention and/or treatment of a tauopathy in a subject in need thereof. Thus, in an embodiment, the invention provides a kit of parts comprising a compound of Formula (I) or a stereoisomeric for thereof, or a pharmaceutically acceptable salt or a solvate thereof, or a pharmaceutical composition comprising said compound, and instructions for preventing or treating a tauopathy. The kit referred to herein can be, in particular, a pharmaceutical package suitable for commercial sale.

For the compositions, methods and kits provided above, one of skill in the art will understand that preferred compounds for use in each are those compounds that are noted as preferred above. Still further preferred compounds for the compositions, methods and kits are those compounds provided in the non-limiting Examples below.

### Experimental Part

Hereinafter, the term “m.p.” means melting point, “min” means minutes, “ACN” means acetonitrile, “aq.” means aqueous, “Boc” means tert-butyloxycarbonyl, “DIAD” means diisopropylazodicarboxylate, “DMF” means dimethylformamide, “r.t.” or “RT” means room temperature, “rac” or “RS” means racemic, “sat.” means saturated, “SFC” means supercritical fluid chromatography, “SFC-MS” means supercritical fluid chromatography/mass spectrometry, “LC-MS” means liquid chromatography/mass spectrometry, “HPLC” means high-performance liquid chromatography, “¹PrOH” means isopropyl alcohol, “RP” means reversed phase, “Rt” means retention time (in minutes), “[M+H]⁺” means the protonated mass of the free base of the compound, “wt” means weight, “THF” means tetrahydrofuran, “Et₂O” means diethylether, “EtOAc” means ethyl acetate, “DCM” means dichloromethane, “MeOH” means methanol, “sat” means saturated, “soltn” or “sol.” means solution, “EtOH” means ethanol, “TFA” means trifluoroacetic acid, “2-meTHF” means 2-methyl-tetrahydrofuran, “Pd(OAc)₂” or “(OAc)₂Pd” means palladium(II) acetate, and “Pd₂(dba)₃” means tris(dibenzylideneacetone)dipalladium(0).

Whenever the notation “RS” is indicated herein, it denotes that the compound is a racemic mixture at the indicated centre, unless otherwise indicated. The stereochemical configuration for centres in some compounds has been designated “R” or “S” when the mixture(s) was separated; for some compounds, the stereochemical configuration at indicated centres has been designated as “R*” or “S*” when the absolute stereochemistry is undetermined although the compound itself has been isolated as a single stereoisomer and is enantiomerically/diastereomerically pure. The enantiomeric excess of compounds reported herein was determined by analysis of the racemic mixture by supercritical fluid chromatography (SFC) followed by SFC comparison of the separated enantiomer(s).

Flow chemistry reactions were performed in a Vapourtec R2+R4 unit using standard reactors provided by the vendor.

Microwave assisted reactions were performed in a single-mode reactor: Initiator™ Sixty EXP microwave reactor (Biotage AB), or in a multimode reactor: MicroSYNTH Labstation (Milestone, Inc.).

Thin layer chromatography (TLC) was carried out on silica gel 60 F254 plates (Merck) using reagent grade solvents. Open column chromatography was performed on silica gel, particle size 60 Å, mesh=230-400 (Merck) using standard techniques. Automated flash column chromatography was performed using ready-to-connect cartridges, on irregular silica gel, particle size 15-40 μm (normal phase disposable flash columns) on different flash systems: either a SPOT or LAFLASH systems from Armen Instrument, or PuriFlash® 430evo systems from Interchim, or 971-FP systems from Agilent, or Isolera 1SV systems from Biotage.

### Preparation of the Intermediates

### Preparation of Intermediate 1

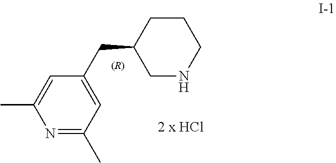

A 2-MeTHF (182.6 mL) solution of intermediate 2 (18.26 g, 59.98 mmol) was charged to a 400 mL reactor equipped with overhead stirrer under nitrogen. The resulting clear orange solution was cooled down to 0° C. and HCl (149.9 mL, 599.8 mmol, 4M solution in 1,4-dioxane) was added dropwise, maintaining the internal temperature below 5° C. The reaction mixture was stirred for 30 min at this temperature and warmed to 20° C. afterwards. A solid (bis HCl salt) crystallized with time. After 1 h at 20° C., the slurry was warmed to 50° C. and stirred for an extra 2 h. After that time, contents were cooled down to 0° C. and slurry filtered off. The wet cake was washed with 2-MeTHF (50 mL) and dried under vacuum at 50° C. overnight to yield intermediate 1 (16.18 g, 97%, 2× HCl salt) as a white solid.

### Preparation of Intermediate 2

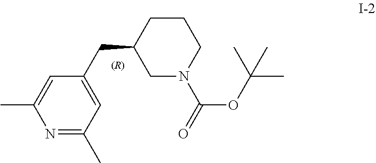

To a 400 mL reactor equipped with overhead stirrer and temperature probe, 4-bromo-2,6-dimethylpyridine (21 g, 113 mmol) was charged under N2 atmosphere at rt. A THF solution of intermediate 3 (366 mL, 124.44 mmol, 0.34 M solution in THF) was then added followed by N,N,N′,N′-tetramethylethylenediamine (18.66 mL, 124.4 mmol) and contents were degassed by N2 sparging (5 min).

Bis(triphenylphosphine)palladium(II) dichloride (CAS: 13965-03-2; 1.588 g, 2.263 mmol) was then added and contents degassed again by N2 sparging for another 5 min. After this, the reaction mixture was warmed to 50° C. and stirred at this temperature for 1 h. The reaction mixture was then cooled down to 20° C. and quenched with a 1:1 mixture of 32% aq. NH₃ and sat. NH₄Cl (200 mL). Water (100 mL) was added followed by EtOAc (200 mL). The resulting biphasic solution was filtered through a pad of Celite® to remove the palladium black residue. Phases were then separated and the aqueous was extracted with EtOAc (200 mL). The combined organic extracts were dried over MgSO₄, solids filtered and solvents distilled under reduced pressure to dryness. Crude material was purified by normal phase column chromatography (silica, EtOAc in heptane 0/100 to 50/50). Desired fractions were collected and concentrated under reduced pressure to yield intermediate 2 (34.44 g, 89% yield) as an orange oil.

### Preparation of Intermediate 3

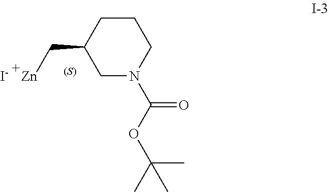

A solution of 3S-iodomethylpiperidine-1-carboxylic acid tert-butyl ester (CAS: 384829-99-6; 47.9 g, 147.3 mmol) in THF (292.8 mL) was pumped through a column containing activated zinc (14.45 g, 221 mmol) at 40° C. under N2 at a flow rate of 1.5 mL/min. The resulting solution was collected over molecular sieves under N2 atmosphere to yield intermediate 3 as a clear light brown solution. This solution was titrated twice against iodine in THF (0.34 M) and used as such in the next step.

### Preparation of Intermediate 4

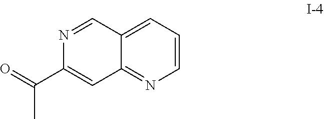

Bis(triphenylphosphine)palladium(II) chloride (53 mg, 0.076 mmol) was added to a stirred suspension of 1-ethoxy-1-(tributylstannyl)ethylene (CAS: 97674-02-7; 302 mg, 0.83 mmol) and intermediate 5 (212 mg, 0.76 mmol) in toluene (4 mL) under N2 atmosphere in a sealed tube. Then the mixture was stirred at 100° C. for 16 h. Then HCl (1.14 mL, 2M solution in diethyl ether) was added and the mixture was stirred at rt for 16 h. Then the mixture was neutralized with NaHCO₃ (aq sat soltn) and extracted with EtOAc. The organic layer was separated, dried (Na₂SO₄), filtered and concentrated in vacuo. The residue was purified by flash column chromatography (SiO₂, MeOH in DCM from 0/100 to 10/90). The desired fractions were collected and concentrated in vacuo. The residue was purified by ion exchange chromatography (ISOLUTE SCX2, eluting with MeOH and 7N ammonia solution in MeOH). The desired fraction was concentrated in vacuo to intermediate 4 (92 mg, 62% pure) as a brown solid.

### Preparation of Intermediate 5

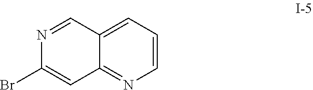 [SMILES: Brc1cc2ncccc2cn1]

Tributyltin hydride (0.55 mL, 2.05 mmol) was added to a stirred suspension of 7-bromo-2-chloro-1,6-naphthyridine (CAS:1578484-42-0; 500 mg, 2.05 mmol) and Pd(PPh₃)₄ (200 mg, 0.17 mmol) in toluene (29 mL) under N2 atmosphere in a sealed tube at rt. The mixture was stirred at rt for 16 h. Then the mixture was diluted with EtOAc and washed with water. The organic layer was separated, dried (Na₂SO₄), filtered and concentrated in vacuo. The residue was purified by flash chromatography (SiO₂, EtOAc in heptane from 0/100 to 100/0). The desired fractions were collected and concentrated in vacuo to intermediate 5 (572 mg, 75% pure) as a pale yellow solid.

### Preparation of Intermediate 6

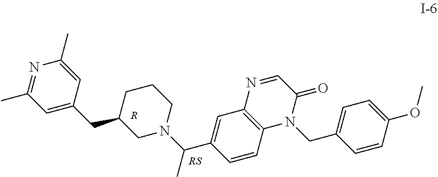

A solution of intermediate 1 (90 mg, 0.44 mmol) in MeOH (2.5 mL) followed by titanium(IV) isopropoxide (0.178 mL, 0.6 mmol) and sodium cyanoborohydride (85 mg, 1.35 mmol) were added to intermediate 7 (186 mg, 0.6 mmol) in a sealed tube and under N2 atmosphere. The mixture was stirred at 80° C. for 40 h. The solvent was evaporated in vacuo and the crude product was purified by flash column chromatography (SiO₂, 7N solution of NH₃ in MeOH in DCM 0/100 to 10/90) and by RP HPLC (stationary phase: C18 XBridge 30×100 mm 5 μm), mobile phase: gradient from 67% NH₄HCO₃ 0.25% solution in water, 33% CH₃CN to 50% NH₄HCO₃ 0.25% solution in water, 50% CH₃CN). The desired fractions were collected and concentrated in vacuo to yield intermediate 6 (29 mg, 13%) as a pale yellow foam.

### Preparation of Intermediate 7

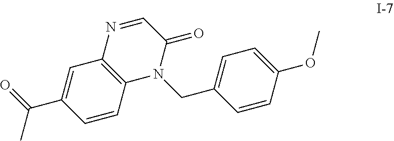 [SMILES: COc1ccc(Cn2c(=O)cnc3cc(C(C)=O)ccc32)cc1]

Tributyl(1-ethoxyvinyl)tin (CAS:97674-02-7; 0.85 mL, 2.52 mmol) followed by bis(triphenylphosphine)palladium(II) chloride (130 mg, 0.19 mmol) were added to a stirred solution of intermediate 8 (726 mg, 2.1 mmol) in toluene (10 mL) in a sealed tube and under N2 atmosphere. The mixture was stirred at 80° C. for 16 h. Then HCl (4.5 mL, 1 M solution in diethyl ether) was added and the mixture was stirred at 80° C. for 3 h. The mixture was added to a stirred solution of sat NaHCO₃ and ice and extracted with DCM. The organic layer was separated, dried (MgSO₄), filtered and the solvents evaporated in vacuo. The crude product was purified by flash column chromatography (SiO₂, EtOAc in heptane 0/100 to 100/0). The desired fractions were collected and concentrated in vacuo to yield intermediate 7 (400 mg, 62%) as a pale yellow solid.

### Preparation of Intermediate 8

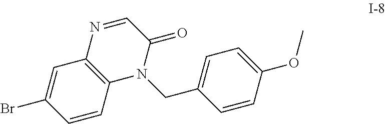 [SMILES: COc1ccc(Cn2c(=O)cnc3cc(Br)ccc32)cc1]

Potassium carbonate (668 mg, 4.83 mmol) followed by sodium iodide (77 mg, 0. 51 mmol) were added portionwise, and subsequently, 4-methoxybenzyl chloride (0.55 mL, 4.05 mmol) was added dropwise to a stirred suspension of 6-bromo-2-hydroxyquinoxaline (813 mg, 3.61 mmol) in DMF (18 mL) in a sealed tube and under N2 atmosphere at rt. The mixture was stirred at rt for 16 h. The mixture was treated with water and extracted with EtOAc. The organic layer was separated, dried (MgSO₄), filtered and the solvents evaporated in vacuo. The crude product was purified by flash column chromatography (SiO₂, EtOAc in heptane 0/100 to 50/50). The desired fractions were collected and concentrated in vacuo to yield intermediate 8 (726 mg, 58%) as a white solid.

### Preparation of Final Compounds

### E1. Preparation of Product 1

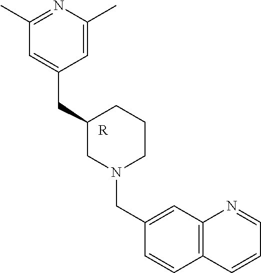

Sodium triacetoxy borohydride (286 mg, 1.35 mmol) was added to a mixture of intermediate 1 (250 mg, 0.9 mmol), 7-quinolinecarbaldehyde (147 mg, 0.94 mmol) and triethylamine (0.376 mL, 2.71 mmol) in MeOH (2.9 mL). The reaction mixture was stirred at rt for 16 h, then water was added and extracted with EtOAc. The organic layer was separated, dried (Na₂SO₄), filtered and concentrated in vacuo. The resultant oil was purified by flash column chromatography (silica; 7M solution of ammonia in methanol in DCM 0/100 to 07/91). The desired fractions were collected and concentrated in vacuo to give a residue that was purified by RP HPLC (stationary phase: C18 XBridge 30×100 mm 5 m), mobile phase: gradient from 81% 10 mM NH₄CO₃H pH 7.9 solution in Water, 19% CH₃CN to 64% 10 mM NH₄CO₃H pH 7.9 solution in Water, 36% CH₃CN). The desired fractions were collected and the solvents evaporated in vacuo to yield product 1 (180 mg, 58%) as a white solid.

### E2. Preparation of Products 2 and 3

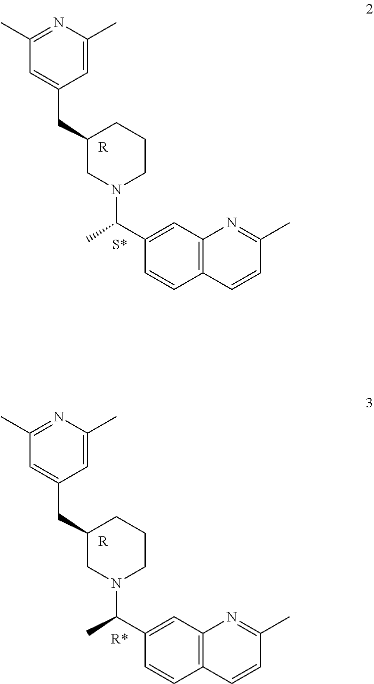

Titanium(IV) isopropoxide (63 μL) was added to a stirred solution of intermediate 1 (53.3 mg, 0.27 mmol) and 2-methyl-7-acetyl-quinoline (CAS: 168083-43-0; 93 mg, 0.5 mmol) in MeOH (2.62 mL) at rt and under N2 atmosphere. The mixture was stirred in a sealed tube at 130° C. for 30 min under microwave irradiation. Then additional titanium(IV) isopropoxide (200 μL) and 2-methyl-7-acetyl-quinoline (CAS: 168083-43-0; 22 mg) were added and the mixture was stirred in a sealed tube at 130° C. for 30 min under microwave irradiation. The solvents were evaporated in vacuo. The crude product was purified by flash column chromatography (silica; 7N solution of ammonia in methanol in DCM 0/100 to 10/90). The desired fractions were collected and the solvents evaporated in vacuo to yield a mixture if product 2 and product 3. This mixture was further purified by RP HPLC (stationary phase: C18 XBridge 30×100 mm 5 μm), mobile phase: gradient from 60% NH₄CO₃H 0.25% solution in water, 40% CH₃CN to 43% NH₄CO₃H 0.25% solution in water, 57% CH₃CN). The desired fractions were collected and concentrated in vacuo to yield product 2 (22.5 mg, 12%) and product 3 (18.6 mg, 10%) as yellow oils.

### E3. Preparation of Products 4 and 5

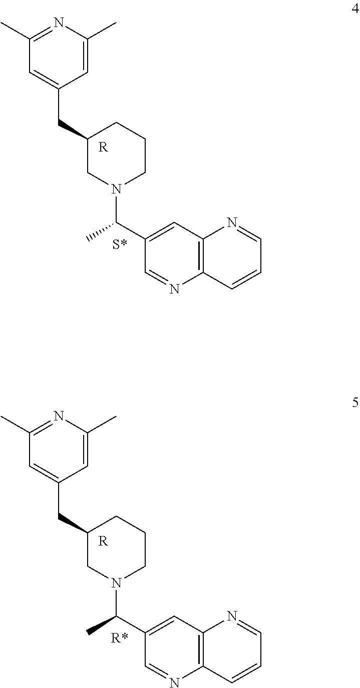

Product 4 and product 5 were prepared following an analogous procedure to the one described for the synthesis of product 2 using intermediate 1 (56 mg, 0.27 mmol) and 1-(1,5-naphthyridin-3-yl)ethanone (CAS: 1246088-62-9) as starting materials. Product 4 and product 5 were purified by RP HPLC (stationary phase: C18 XBridge 30×100 mm 5 μm), mobile phase: gradient from 80% NH₄CO₃H 0.25% solution in water, 20% CH₃CN to 60% NH₄CO₃H 0.25% solution in water, 40% CH₃CN). The desired fractions were collected and concentrated in vacuo to yield product 4 (10 mg, 10%, mixture of diastereoisomers 85:15 being product 5 the minor one) and product 5 (5.9 mg, 6%) as yellow oils.

### E4. Preparation of Product 6

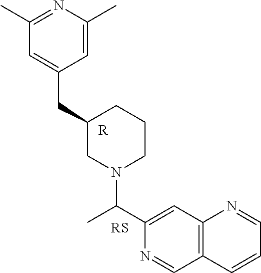

Triethylamine (0.14 mL, 1 mmol) was added to a stirred suspension of intermediate 1 (92 mg, 0.33 mmol) and intermediate 4 (92 mg, 0.33 mmol) in DCM (1.9 mL) in sealed tube at rt and the mixture was stirred at rt for 5 min (until disolution). Then sodium cyanoborhydride (89 mg, 1.42 mmol) and titanium(IV) isopropoxide (0.19 mL, 0.64 mmol) were added and the mixture was stirred at 80° C. for 2 h. Then the mixture was concentrated in vacuo and the residue thus obtained was purified by flash column chromatography (silica; 7M ammonia solution in methanol in DCM 0/100 to 10/90). The desired fractions were collected and concentrated in vacuo. The residue was purified by RP HPLC (stationary phase: XBridge C18 50×100 mm, 5 μm), mobile phase: gradient from 80% NH₄CO₃H 0.25% solution in water, 20% CH₃CN to 63% NH₄CO₃H 0.25% solution in water, 37% CH₃CN). The desired fractions were collected and concentrated in vacuo to yield product 6 (12 mg, mixture of diastereoisomers) as a pale yellow oil.

### E5. Preparation of Product 7

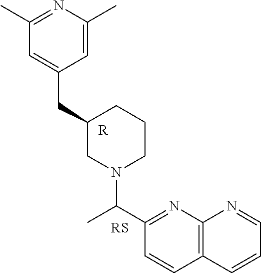

Product 7 was prepared following an analogous procedure to the one described for the synthesis of product 6 using intermediate 1 (117.1 mg, 0.42 mmol) and 1-(1,8-naphthyridin-2-yl)ethan-1-one (CAS: 1188433-77-3) as starting materials. Product 7 was purified by RP HPLC (stationary phase: YMC 40 g, 25 μm), mobile phase: gradient from 30% NH₄CO₃H 0.25% solution in water, 70% MeOH to 0% NH₄CO₃H 0.25% solution in water, 100% MeOH). The desired fractions were collected and concentrated in vacuo to yield product 7 (107 mg, 70%, mixture of diastereoisomers) as a yellow oil.

### E6. Preparation of Product 8

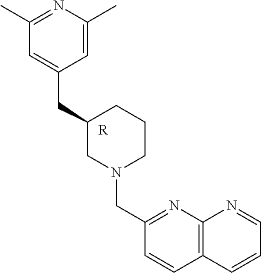

Product 8 was prepared following an analogous procedure to the one described for the synthesis of product 1 using intermediate 1 (100 mg, 0.36 mmol) and 1,8-naphthyridin-2-carbaldehyde (CAS: 64379-45-9) as starting materials. Product 8 was purified by RP HPLC (stationary phase: XBridge C18 30×100 mm, 5 μm), mobile phase: gradient from 80% NH₄CO₃H 0.25% solution in water, 60% CH₃CN to 60% NH₄CO₃H 0.25% solution in water, 40% CH₃CN). The desired fractions were collected and concentrated in vacuo to yield impure product 8 (75 mg) as a yellow oil. Impure product 8 (75 mg) was dissolved in MeOH (2 mL) and HCl (0.5 mL, 6N solution in i-PrOH) was added. The mixture was concentrated in vacuo to yield impure product 8 (104 mg, 3× HCl salt) as a brown solid. Impure product 8 (104 mg, 3×HCl salt) was purified by ion exchange chromatography using an ISOLUTE SCX2 cartridge eluting first with MeOH and then with 7N solution of ammonia in methanol. The desired fraction was collected, concentrated in vacuo, re-dissolved in MeOH (2 mL) and HCl (0.5 mL, 6N solution in i-PrOH) was added. The mixture was concentrated in vacuo to yield product 8 (63 mg, 38%, 3×HCl salt) as a brown solid.

### E7. Preparation of Product 9

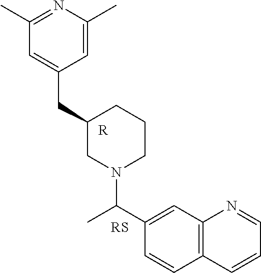

Product 9 was prepared following an analogous procedure to the one described for the synthesis of product 6 using intermediate 1 (250 mg, 0.9 mmol) and 1-(7-quinolinyl)ethanone (CAS: 103854-57-5) as starting materials. Product 9 was purified by purified by ion exchange chromatography using an ISOLUTE SCX2 cartridge eluting first with MeOH and then with 7N solution of ammonia in methanol. The desired fraction was collected, concentrated in vacuo to give a residue that was further purified by RP HPLC (stationary phase: XBridge C18 30×100 mm, 5 μm), mobile phase: gradient from 74% 10 mM NH₄CO₃H pH 7.9 solution in water, 26% CH₃CN to 58% 10 mM NH₄CO₃H pH 7.9 solution in water, 42% CH₃CN). The desired fractions were collected and concentrated in vacuo to yield product 9 as an oil. Product 9 was dissolved in MeOH and HCl (6N solution in i-PrOH) was added. The mixture was concentrated in vacuo to yield product 9 (220 mg, 56%, mixture of diastereoisomers, 2× HCl salt) as a white solid.

### E8. Preparation of Product 10

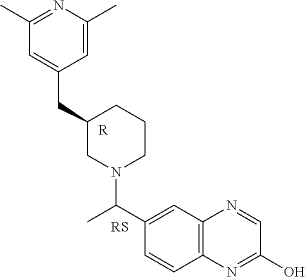

Trifluoroacetic acid (0.8 mL) was added to intermediate 6 (36 mg, 0.072 mmol) in a sealed tube and under N2 atmosphere. The mixture was stirred at 80° C. for 16 h. The solvent was evaporated in vacuo. The crude product was purified by ion exchange chromatography (ISOLUTE SCX-2, MeOH and then 7N solution of NH₃ in MeOH) and by flash column chromatography (SiO₂, 7N solution of NH₃ in MeOH in DCM 0/100 to 10/90). The desired fractions were collected and concentrated in vacuo to yield product 10 (16 mg, 58%) as a dark yellow oil.

The following compounds were prepared following the methods exemplified in the Experimental Part. In case no salt form is indicated, the compound was obtained as a free base. ‘Ex. No.’ refers to the Example number according to which protocol the compound was synthesized. ‘Co. No.’ means compound number.

TABLE 1

| | |
|---|---|
| | (I) |
| 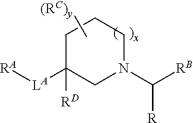 | |

| Co. | Exp. | | Salt |
|---|---|---|---|
| No. | No. | Co. Formula (I) | Form |
| 1 | E1 | 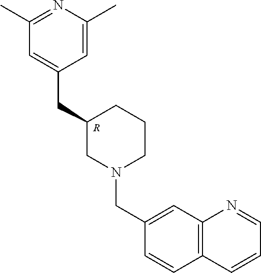 | |
| 2 | E2 | 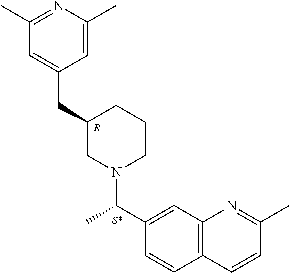 | |
| 3 | E2 | 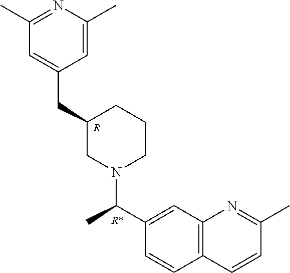 | |
| 4 | E3 | 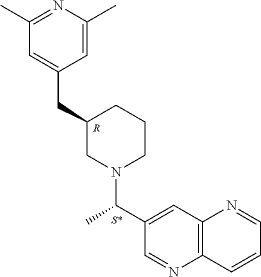 | |
| 5 | E3 | 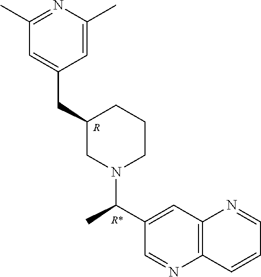 | |
| 6 | E4 | 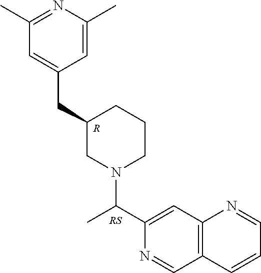 | |
| 7 | E5 | 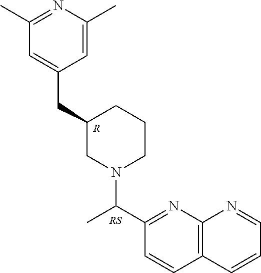 | |
| 8 | E6 | 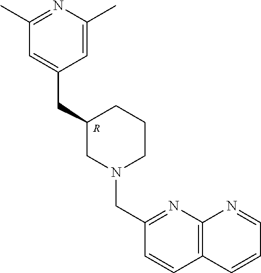 | 3•HCl |
| 9 | E7 | 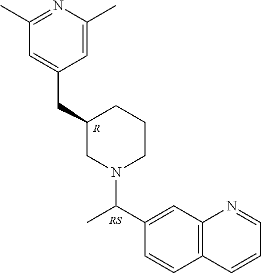 | •2HCl |
| 10 | E8 | 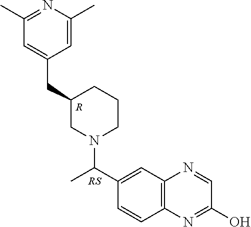 | |

The values of salt stoichiometry or acid content in the compounds as provided herein, are those obtained experimentally. The content of hydrochloric acid reported herein was determined by ¹H NMR integration and/or elemental analysis.

### Analytical Part

### Melting Points

Values are peak values, and are obtained with experimental uncertainties that are commonly associated with this analytical method.

DSC823e: For a number of compounds, melting points were determined with a DSC823e (Mettler-Toledo) apparatus. Melting points were measured with a temperature gradient of 10° C./minute. Maximum temperature was 300° C. Values are peak values (A).

### LCMS

### General Procedure

The High Performance Liquid Chromatography (HPLC) measurement was performed using a LC pump, a diode-array (DAD) or a UV detector and a column as specified in the respective methods. If necessary, additional detectors were included (see table of methods below).

Flow from the column was brought to the Mass Spectrometer (MS) which was configured with an atmospheric pressure ion source. It is within the knowledge of the skilled person to set the tune parameters (e.g. scanning range, dwell time . . . ) in order to obtain ions allowing the identification of the compound's nominal monoisotopic molecular weight (MW) and/or exact mass monoisotopic molecular weight. Data acquisition was performed with appropriate software.

Compounds are described by their experimental retention times (Rₜ) and ions. If not specified differently in the table of data, the reported molecular ion corresponds to the [M+H]⁺ (protonated molecule) and/or [M−H]⁻ (deprotonated molecule). In case the compound was not directly ionizable the type of adduct is specified (i.e. [M+NH₄]⁺, [M+HCOO]⁻, [M+CH₃COO]⁻ etc. . . . ). For molecules with multiple isotopic patterns (Br, Cl.), the reported value is the one obtained for the lowest isotope mass. All results were obtained with experimental uncertainties that are commonly associated with the method used.

Hereinafter, “SQD” Single Quadrupole Detector, “MSD” Mass Selective Detector, “QTOF” Quadrupole-Time of Flight, “rt” room temperature, “BEH” bridged ethylsiloxane/silica hybrid, HSS” High Strength Silica, “CSH” charged surface hybrid, “UPLC” Ultra Performance Liquid Chromatography, “DAD” Diode Array Detector.

TABLE 2
LC-MS Method (Flow expressed in mL/min; column temperature (T) in ° C.; Run time in min).

| | | | | | Flow | |
|---|---|---|---|---|---|---|
| Method | | | | | Col | Run |
| code | Instrument | Column | Mobile phase | Gradient | T | time |
| 1 | Waters: | Waters: BEH C18 | A: 95% | From | 1 | 5 |
| | Acquity ® | (1.7 μm, 2.1 × 50 mm) | CH₃COONH₄ | 95% A | −50 | |
| | IClass | | 6.5 mM + 5% | to 5% A | | |
| | UPLC ®- | | CH₃CN, B: | in | | |
| | DAD and | | CH₃CN | 4.6 min, | | |
| | Xevo G2-S | | | held for | | |
| | QTOF | | | 0.4 min | | |

TABLE 3
Analytical data-melting point (M.p.) and LCMS: [M + H]⁺ means the
protonated mass of the free base of the compound, [M − H]⁻ means the
deprotonated mass of the free base of the compound or the type of adduct
specified [M + CH₃COO]⁻). Rₜ means retention time (in min).
For some compounds, exact mass was determined.

| Co. | M.p. | | | LCMS |
|---|---|---|---|---|
| No. | (° C.) | [M + H]⁺ | Rₜ | Method |
| 1 | 152.83 | 346 | 1.75 | 1 |
| 2 | n.d. | 374 | 1.83 | 1 |
| 3 | n.d. | 374 | 1.93 | 1 |
| 4 | n.d. | 361 | 1.68 | 1 |
| 5 | n.d. | 361 | 1.70 | 1 |
| 6 | n.d. | 361 | 1.3 | 1 |
| 7 | n.d. | 361 | 1.30 | 1 |
| 8 | n.d. | 347 | 1.33 | 1 |
| 9 | 262.69 | 360 | 1.74/1.79 | 1 |
| 10 | n.d. | 377 | 1.30 | 1 |

### Optical Rotations

Optical rotations were measured on a Perkin-Elmer 341 polarimeter with a sodium lamp and reported as follows: [α]° (λ, c g/100 ml, solvent, T ° C.).

[α]_{λ}^{T}=(100α)/(l×c): where l is the path length in dm and c is the concentration in g/100 ml for a sample at a temperature T (° C.) and a wavelength λ (in nm). If the wavelength of light used is 589 nm (the sodium D line), then the symbol D might be used instead. The sign of the rotation (+ or −) should always be given. When using this equation, the concentration and solvent are always provided in parentheses after the rotation. The rotation is reported using degrees and no units of concentration are given (it is assumed to be g/100 mL).

TABLE 4
Optical Rotation data.

| Co. | | Wavelength | Concentration | | Temp. |
|---|---|---|---|---|---|
| No. | α_{D} (°) | (nm) | w/v % | Solvent | (° C.) |
| 1 | −4.1 | 589 | 0.53 | MeOH | 20 |
| 8 | +24.9 | 589 | 0.5 | MeOH | 20 |

### Pharmacological Examples

### 1) OGA—Biochemical Assay

The assay is based on the inhibition of the hydrolysis of fluorescein mono-β-D-N-Acetyl-Glucosamine (FM-GlcNAc) (Mariappa et al. 2015, Biochem J 470:255) by the recombinant human Meningioma Expressed Antigen 5 (MGEA5), also referred to as O-GlcNAcase (OGA). The hydrolysis FM-GlcNAc (Marker Gene technologies, cat #M1485) results in the formation of β-D-N-glucosamineacetate and fluorescein. The fluorescence of the latter can be measured at excitation wavelength 485 nm and emission wavelength 538 nm. An increase in enzyme activity results in an increase in fluorescence signal. Full length OGA enzyme was purchased at OnGene (cat #TP322411). The enzyme was stored in 25 mM Tris.HCl, pH 7.3, 100 mM glycine, 10% glycerol at −20° C. Thiamet G and GlcNAcStatin were tested as reference compounds (Yuzwa et al. 2008 Nature Chemical Biology 4:483; Yuzwa et al. 2012 Nature Chemical Biology 8:393). The assay was performed in 200 mM Citrate/phosphate buffer supplemented with 0.005% Tween-20. 35.6 g Na₂HPO₄ 2 H₂O (Sigma, #C0759) were dissolved in 1 L water to obtain a 200 mM solution. 19.2 g citric acid (Merck, #1.06580) was dissolved in 1 L water to obtain a 100 mM solution. pH of the sodiumphosphate solution was adjusted with the citric acid solution to 7.2. The buffer to stop the reaction consists of a 500 mM Carbonate buffer, pH 11.0. 734 mg FM-GlcNAc were dissolved in 5.48 mL DMSO to obtain a 250 mM solution and was stored at −20° C. OGA was used at a 2 nM concentration and FM-GlcNAc at a 100 uM final concentration. Dilutions were prepared in assay buffer.

50 nl of a compound dissolved in DMSO was dispensed on Black Proxiplate™ 384 Plus Assay plates (Perkin Elmer, #6008269) and 3 μl fl-OGA enzyme mix added subsequently. Plates were pre-incubated for 60 min at room temperature and then 2 μl FM-GlcNAc substrate mix added. Final DMSO concentrations did not exceed 1%. Plates were briefly centrifuged for 1 min at 1000 rpm and incubate at room temperature for 6 h. To stop the reaction 5 μl STOP buffer were added and plates centrifuge again 1 min at 1000 rpm. Fluorescence was quantified in the Thermo Scientific Fluoroskan Ascent or the PerkinElmer EnVision with excitation wavelength 485 nm and emission wavelength 538 nm.

For analysis a best-fit curve is fitted by a minimum sum of squares method. From this an IC₅₀ value and Hill coefficient was obtained. High control (no inhibitor) and low control (saturating concentrations of standard inhibitor) were used to define the minimum and maximum values.

### 2) OGA—Cellular Assay

HEK293 cells inducible for P301L mutant human Tau (isoform 2N4R) were established at Janssen. Thiamet-G was used for both plate validation (high control) and as reference compound (reference EC₅₀ assay validation). OGA inhibition is evaluated through the immunocytochemical (ICC) detection of O-GlcNAcylated proteins by the use of a monoclonal antibody (CTD110.6; Cell Signaling, #9875) detecting 0-GlcNAcylated residues as previously described (Dorfmueller et al. 2010 Chemistry & biology, 17:1250). Inhibition of OGA will result in an increase of O-GlcNAcylated protein levels resulting in an increased signal in the experiment. Cell nuclei are stained with Hoechst to give a cell culture quality control and a rough estimate of immediate compounds toxicity, if any. ICC pictures are imaged with a Perkin Elmer Opera Phenix plate microscope and quantified with the provided software Perkin Elmer Harmony 4.1.

Cells were propagated in DMEM high Glucose (Sigma, #D5796) following standard procedures. 2 days before the cell assay cells are split, counted and seeded in Poly-D-Lysine (PDL) coated 96-wells (Greiner, #655946) plate at a cell density of 12,000 cells per cm² (4,000 cells per well) in 100 μl of Assay Medium (Low Glucose medium is used to reduce basal levels of GlcNAcylation) (Park et al. 2014 The Journal of biological chemistry 289:13519). At the day of compound test medium from assay plates was removed and replenished with 90 μl of fresh Assay Medium. 10 μl of compounds at a 10fold final concentration were added to the wells. Plates were centrifuged shortly before incubation in the cell incubator for 6 hours. DMSO concentration was set to 0.2%. Medium is discarded by applying vacuum. For staining of cells medium was removed and cells washed once with 100 μl D-PBS (Sigma, #D8537). From next step onwards unless other stated assay volume was always 50 μl and incubation was performed without agitation and at room temperature. Cells were fixed in 50 μl of a 4% paraformaldehyde (PFA, Alpha aesar, #043368) PBS solution for 15 minutes at room temperature. The PFA PBS solution was then discarded and cells washed once in 10 mM Tris Buffer (LifeTechnologies, #15567-027), 150 mM NaCl (LifeTechnologies, #24740-0110, 0.1% Triton X (Alpha aesar, #A16046), pH 7.5 (ICC buffer) before being permeabilized in same buffer for 10 minutes. Samples are subsequently blocked in ICC containing 5% goat serum (Sigma, #G9023) for 45-60 minutes at room temperature. Samples were then incubated with primary antibody (1/1000 from commercial provider, see above) at 4° C. overnight and subsequently washed 3 times for 5 minutes in ICC buffer. Samples were incubated with secondary fluorescent antibody (1/500 dilution, Lifetechnologies, #A-21042) and nuclei stained with Hoechst 33342 at a final concentration of 1 μg/ml in ICC (Lifetechnologies, #H3570) for 1 hour. Before analysis samples were washed 2 times manually for 5 minutes in ICC base buffer.

Imaging is performed using Perkin Elmer Phenix Opera using a water 20× objective and recording 9 fields per well. Intensity readout at 488 nm is used as a measure of O-GlcNAcylation level of total proteins in wells. To assess potential toxicity of compounds nuclei were counted using the Hoechst staining. IC₅₀-values are calculated using parametric non-linear regression model fitting. As a maximum inhibition Thiamet G at a 200 uM concentration is present on each plate. In addition, a concentration response of Thiamet G is calculated on each plate.

TABLE 5
Results in the biochemical and cellular assays.

| | Enzymatic | | Cellular | Cellular |
|---|---|---|---|---|
| Co. | hOGA; | Enzymatic | hOGA; | Eₘₐₓ |
| No. | pIC₅₀ | Eₘₐₓ (%) | pEC₅₀ | (%) |
| 1 | 6.54 | 95.79 | <6 | 33.04 |
| 2 | 5.14 | 60.76 | nt | nt |
| 3 | 7.27 | 101.10 | <6 | 34.75 |
| 4 | 7.54 | 102.08 | 6.19 | 50.54 |
| 5 | 8.26 | 102.91 | 6.79 | 86.36 |
| 6 | 7.32 | 99.58 | 6 | 41.12 |
| 7 | 7.83 | 101.43 | 6.63 | 71.14 |
| 8 | 6.71 | 98.47 | <6 | 29.75 |
| 10 | 6.97 | 100.89 | <6 | 11.23 |

1. A compound of Formula (I) 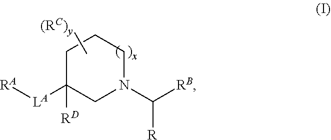
or a tautomer or a stereoisomeric form thereof, wherein
R^{A} is a heteroaryl radical selected from the group consisting of pyridin-2-yl, pyridin-3-yl, pyridin-4-yl, pyridazin-3-yl, pyrimidin-4-yl, pyrimidin-5-yl, and pyrazin-2-yl, each of which may be optionally substituted with 1, 2 or 3 substituents each independently selected from the group consisting of halo; cyano, C₁₋₄alkyl optionally substituted with 1, 2, or 3 independently selected halo substituents; —C(O)NR^{a}R^{aa}; NR^{a}R^{aa}; and C₁₋₄alkyloxy optionally substituted with 1, 2, or 3 independently selected halo substituents; wherein R^{a} and R^{aa} are each independently selected from the group consisting of hydrogen and C₁₋₄alkyl optionally substituted with 1, 2, or 3 independently selected halo substituents;
L^{A} is selected from the group consisting of a covalent bond, —O—, —CH₂—, —OCH₂—, —CH₂O—, —NH—, —N(CH₃)—, —NH—CH₂—, and —CH₂—NH—;
x represents 0 or 1;
R is H or CH₃; and
R^{B} is an aromatic heterobicyclic radical selected from the group consisting of (b-1), (b-2), (b-3) and (b-4) 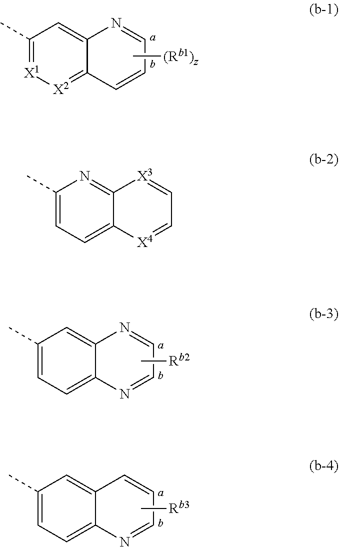
wherein
X¹ and X² are each independently selected from CH and N, with the proviso that at least one is CH; z represents 0 or 1; X³ and X⁴ are each independently selected from CH and N, with the proviso that X³ is CH when X⁴ is N, and X³ is N when X⁴ is CH;
R^{b1}, R^{b2}, and R^{b3} are each independently selected from the group consisting of methyl, hydroxy, and halo;
a and b represent the optional position of attachment for R^{b1}, R^{b2}, or R^{b3};
R^{C} is selected from the group consisting of fluoro, methyl, hydroxy, methoxy, trifluoromethyl, and difluoromethyl;
R^{D} is selected from the group consisting of hydrogen, fluoro, methyl, hydroxy, methoxy, trifluoromethyl, and difluoromethyl; and
y represents 0, 1 or 2;
with the provisos that
a) R^{C} is not hydroxy or methoxy when present at the carbon atom adjacent to the nitrogen atom of the piperidinediyl or pyrrolidinediyl ring;
b) R^{C} or R^{D} cannot be selected simultaneously from hydroxy or methoxy when R^{C} is present at the carbon atom adjacent to C—R^{D}; and
c) R^{D} is not hydroxy or methoxy when L^{A} is —O—, —OCH₂—, —CH₂O—, —NH—, —N(CH₃)—, —NHCH₂— or —CH₂NH—;
or a pharmaceutically acceptable addition salt or a solvate thereof.

2. The compound according to claim 1, wherein R is (b-1), (b-2) or (b-3), wherein X¹ and X² are both CH; or X¹ is CH and X² is N; or X¹ is N and X² is CH; z is 0 or 1; R^{b1} is CH₃; X³ is N and X⁴ is CH; and R^{b2} is OH.

3. The compound according to claim 1, wherein y is 0 and R^{D} is hydrogen.

4. The compound according to claim 1, wherein R^{A} is pyridin-4-yl; optionally substituted with 1 or 2 substituents, each independently selected from the group consisting of halo; C₁₋₄alkyl optionally substituted with 1, 2, or 3 independently selected halo substituents; and C₁₋₄alkyloxy optionally substituted with 1, 2, or 3 independently selected halo substituents; more in particular, R^{A} is pyridin-4-yl substituted with 1 or 2 independently selected C₁₋₄alkyl substituents.

5. The compound according to claim 1, wherein L^{A} is —CH₂—.

6. The compound according to claim 1, wherein R^{B} is selected from the group consisting of

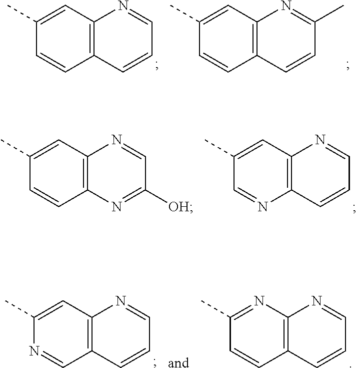

7. The compound according to any one of claim 1, wherein R^{B} is

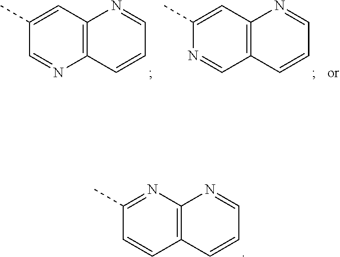

8. The compound according to claim 1, wherein R^{A} is

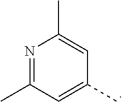

9. A pharmaceutical composition comprising a prophylactically or a therapeutically effective amount of a compound according to claim 1 and a pharmaceutically acceptable carrier.

10. (canceled)

11. (canceled)

12. (canceled)

13. A method of preventing or treating a disorder selected from the group consisting of tauopathy, in particular a tauopathy selected from the group consisting of Alzheimer's disease, progressive supranuclear palsy, Down's syndrome, frontotemporal lobe dementia, frontotemporal dementia with Parkinsonism-17, Pick's disease, corticobasal degeneration, and agryophilic grain disease; or a neurodegenerative disease accompanied by a tau pathology, in particular a neurodegenerative disease selected from amyotrophic lateral sclerosis or frontotemporal lobe dementia caused by C9ORF72 mutations, comprising administering to a subject in need thereof, a prophylactically or a therapeutically effective amount of a compound according to claim 1.

14. (canceled)
